(19) United States
(12) Patent Application Publication
(10) Pub. No.: US 2024/0238229 A1
(43) Pub. Date: Jul. 18, 2024

(54) TREATMENT OF PULMONARY ARTERIAL HYPERTENSION

(71) Applicant: United Therapeutics Corporation, Silver Spring, MD (US)

(72) Inventors: Gil Golden, Durham, NC (US); Yan Liu, Durham, NC (US)

(73) Assignee: United Therapeutics Corporation, Silver Spring, MD (US)

(21) Appl. No.: 18/416,054

(22) Filed: Jan. 18, 2024

Related U.S. Application Data
(60) Provisional application No. 63/439,779, filed on Jan. 18, 2023.

(51) Int. Cl. A61K 31/192; A61K 9/00; A61K 31/343; A61P 9/12

(52) U.S. Cl. CPC A61K 31/192; A61K 9/0053; A61K 31/343; A61P 9/12

(57) ABSTRACT

Provided are novel methods of treating pulmonary hypertension comprising monitoring mean pulmonary arterial pressure in a subject, administering a first therapeutically effective amount of a pulmonary vasodilator when mean pulmonary pressure exceeds a threshold value in the subject, and administering an increasing dose of the pulmonary vasodilator until mean pulmonary arterial pressure is reduced.

### CROSS-REFERENCE TO RELATED APPLICATIONS

This application claims priority to U.S. Provisional Application 63/439,779, filed Jan. 18, 2023, the entire contents of which are incorporated herein by reference.

### TECHNICAL FIELD

The present application generally relates to the treatment of pulmonary hypertension.

### BACKGROUND

Pulmonary arterial hypertension (PAH) is a rapidly progressing disease whereby elevated mean pulmonary artery pressure (mPAP>20 mmHg) leads to right ventricular (RV) dysfunction and death. Pulmonary arterial hypertension is characterized by increased pressure in the pulmonary vasculature that can lead to, inter alia, heart failure. (2022 ESC/ERS guidelines for the diagnosis and treatment of pulmonary hypertension. Eur Heart J. 2022;43(38):3618-373)

Pulmonary Hypertension has Classified into Five Groups:
Group 1: pulmonary arterial hypertension (PAH);
Group 2: PH associated with left heart disease;
Group 3: PH associated with lung disease and/or hypoxia;
Group 4: PH associated with pulmonary artery obstructions;
Group 5: PH with unclear and/or multifactorial mechanisms

There are currently a number of approved products for certain types of pulmonary hypertension, including Group 1 (PAH). Those products include products containing treprostinil as the active ingredient, such as Remodulin® (treprostinil) injection. It is generally desirable to dose patients with the maximum tolerated dose of treprostinil to achieve maximum therapeutic effect balanced against undesirable effects, e.g., site pain when administered subcutaneously. A need exists for administering treprostinil to maximize therapeutic effect and minimize undesirable side effects.

A pilot study has demonstrated the feasibility and safety of using CardioMEMS™ HF System to remotely monitor mPAP and guide medical therapy in PAH (Am. J. Respir. Crit. Care Med., 2022 Apr. 1; 205(7):751-760), while certain studies have evaluated early aggressive parenteral prostanoid therapy and shown marked mPAP reduction can lead to significant improvement of RV function and long-term outcomes in patients with PAH (J. Heart Lung Transplant, 2018; 37:365-375).

### SUMMARY

One aspect of the present disclosure is directed to a method of treating pulmonary hypertension comprising monitoring mean pulmonary arterial pressure in a subject and administering to the subject a first therapeutically effective amount of a pulmonary vasodilator when mean pulmonary arterial pressure exceeds a threshold value in the subject. The method may further comprise administering an increasing dose of the pulmonary vasodilator until mean pulmonary arterial pressure is reduced to a target level. In some embodiments, the subject is a human.

In some aspects of the present disclosure, the pulmonary vasodilator comprises a prostanoid or prostacyclin. In yet another aspect, the pulmonary vasodilator comprises treprostinil, a salt or ester thereof. In yet another aspect, the pulmonary vasodilator comprises a non-prostanoid IP receptor agonist such as ralinepag.

In some aspects of the present disclosure, pulmonary arterial pressure in the subject is remotely monitored. In yet another aspect, the mean pulmonary arterial pressure is continuously monitored. In some aspects, mean pulmonary artery pressure is monitored using an implanted wireless pulmonary artery pressure sensor in the subject.

In some aspects of the present disclosure, the target level is less than 50 mm Hg, or less than 40 mm Hg, or less than 30 mm H, or less than 25 mm Hg. In yet further aspects, the threshold value is greater than 20 mm Hg, or greater than 30 mm Hg, or greater than 40 mm Hg. In some aspects, initial mean pulmonary arterial pressure is greater than 35 mm Hg.

In some aspects of the present disclosure, the methods comprise monitoring the right ventricle structure of the subject. In yet another aspect, the method further comprises measuring right ventricular ejection fraction after a first period of time following administration of a pulmonary vasodilator.

In some aspects, ejection fraction is measured by cMRI. In yet another aspect, the method further comprises, consists of or consists essentially of measuring a ratio of stroke volume to end systolic volume. In yet another aspect, the method comprises, consists of or consists essentially of measuring a ratio of tricuspid annular plane systolic excursion to pulmonary artery systolic pressure.

In some aspects of the present disclosure, the subject has not been previously treated for pulmonary hypertension or has received an endothelin receptor antagonist and/or a phosphodiesterase type 5 inhibitor for less than 24 months, or less than 18 months, or less than 12 months, or less than 6 months, or less than 3 months, or less than 1 month.

In some aspects of the present disclosure, administering comprises administering intravenously. In yet another aspect, administering comprises administering by inhalation. In yet another aspect, administering comprises administering an oral formulation.

### DETAILED DESCRIPTION

As used herein and in the claims, the singular forms “a,” “an,” and “the” include the plural reference unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers. The term “or” is inclusive unless modified, for example, by “either.” Thus, unless context indicates otherwise, the word “or” means any one member of a particular list and also includes any combination of members of that list. Other than in the operating examples, or where otherwise indicated, all numbers expressing quantities of ingredients or reaction conditions used herein should be understood as modified in all instances by the term “about.”

Headings are provided for convenience only and are not to be construed to limit the invention in any way. Unless defined otherwise, all technical and scientific terms used herein have the same meaning as those commonly understood to one of ordinary skill in the art. The terminology used herein is for the purpose of describing particular embodiments only, and is not intended to limit the scope of the present invention, which is defined solely by the claims. In order that the present disclosure can be more readily understood, certain terms are first defined. Additional definitions are set forth throughout the detailed description.

All numerical designations are approximations which are varied (+) or (−) by increments of 0.05%, 1%, 2%, 5%, 10% or 20%. It is to be understood, although not always explicitly stated that all numerical designations are preceded by the term “about.” It also is to be understood, although not always explicitly stated, that the reagents described herein are merely exemplary and that equivalents of such are known in the art.

Further aspects of the present invention are concerned with methods of treating pulmonary hypertension comprising monitoring mean pulmonary arterial pressure in a subject. Monitoring preferably is conducted at least once per week or at least once per month. In some aspects, the present invention includes the administration of a pulmonary vasodilator to the subject. In some aspects, the pulmonary vasodilator is Treprostinil or its derivatives, or pharmaceutically acceptable salts thereof.

In one embodiment, a method uses treprostinil sodium, currently marketed under various trade names and routes of administration: REMODULIN® (intravenous and subcutaneous), TYVASO® (inhaled), TYVASO DPI® (inhaled), or ORENITRAM® (oral). The US FDA has approved treprostinil sodium for the treatment pulmonary arterial hypertension by injection of dose concentrations of 1.0 mg/mL, 2.5 mg/mL, 5.0 mg/mL and 10.0 mg/ml, as well as by inhalation and oral administration in the approved products listed above. The chemical structure formula for treprostinil sodium is:

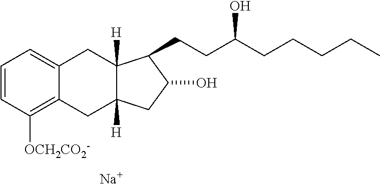

Treprostinil sodium is sometimes designated by the chemical names: (a) [(1R,2R,3aS,9aS)-2,3,3a,4,9,9a-hexahydro-2-hydroxy-1-[(3S)-3-hydroxyoctyl]-1H-benz[f]inden-5-yl]oxy]acetic acid; or (b) 9-deoxy-2′,9-□-methano-3-oxa-4,5,6-trinor-3,7-(1′,3′-interphenylene)-13,14-dihydro-prostaglandin F1. Treprostinil sodium is also known as: UT-15; LRX-15; 15AU81; UNIPROST™; BW A15AU; and U-62,840. The molecular weight of Treprostinil sodium is 390.52, and its empirical formula is C₂₃H₃₄O₅. Treprostinil, or 9-deoxy-2 ‘, 9-alpha-methane-3-oxa-4,5,6-trinor-3,7-(1′3’-interphenylene)-13,14-dihydro-prostaglandin FI, is an analogue of prostacyclin, described for the first time in U.S. Pat. No. 4,306,075. U.S. Pat. No. 5,153,222 describes the use of treprostinil for the treatment of pulmonary hypertension. Treprostinil is approved for the intravenous as well as the subcutaneous route, avoiding the latter the septic effects associated with continuous intravenous catheters. U.S. Pat. Nos. 6,521,212 and 6,756,033 describe the administration of treprostinil by inhalation for the treatment of pulmonary hypertension, peripheral venous insufficiency, and other diseases and conditions. U.S. Pat. No. 6,803,386 describes the administration of treprostinil to treat cancer, such as lung, liver, brain, pancreatic, renal, prostate, breast, colon, and head and neck cancer. US Patent Application Publication No. 2005/0165111 describes the treatment of ischemic lesions with treprostinil. U.S. Pat. No. 7,199,157 discloses that treatment with treprostinil improves renal functions. US Patent Application Publication No. 2005/0282903 describes the treatment of neuropathic foot ulcers with treprostinil. US Provisional Application No. 60/900,320, filed on Feb. 9, 2007, describes the treatment of pulmonary fibrosis with treprostinil.

Physiologically acceptable salts of treprostinil include salts derived from bases. Base salts include ammonium salts (such as quaternary ammonium salts), alkali metal salts such as those of sodium and potassium, alkaline earth metal salts such as those of calcium and magnesium, salts with organic bases such as dicyclohexylamine and N-methyl-D-glucamine, and salts with amino acids such as arginine and lysine. The term “acid derivative” is used herein to describe C₁-4 alkyl esters and amides, including amides in which the nitrogen is optionally substituted with one or two C₁-4 alkyl groups.

Quaternary ammonium salts can be formed, for example, by reaction with lower alkyl halides, such as methyl, ethyl, propyl, and butyl chlorides, bromides, and iodides, with dialkyl sulphates, with long chain halides, such as decyl, lauryl, myristyl, and stearyl chlorides, bromides, and iodides, and with aralkyl halides, such as benzyl and phenethyl bromides.

The amount of treprostinil or its derivative, or a pharmaceutically acceptable salt thereof, that is required in a medication or diagnostic aid according to the invention to achieve the desired effect will depend on a number of factors, such as the specific application, the nature of the particular compound used, the mode of administration, the concentration of the compound used, and the weight and condition of the patient. A concentration of the treprostinil or pharmaceutically acceptable salt thereof in the solution ranges from about 500 pg/ml to about 2500 pg/ml, wherein the metered dose inhaler delivers a single dose in a single actuation of treprostinil or pharmaceutically acceptable salt thereof from 30 pg to 90 pg of treprostinil or the pharmaceutically acceptable salt thereof. Described herein is a method for delivering to a subject in need, such as a human, a therapeutically effective amount of treprostinil, which comprises administering to the subject a formulation comprising a therapeutically effective amount of treprostinil, its derivative, or a pharmaceutically salt acceptable from the same, using a metered dose inhaler. Treprostinil can be administered via a metered dose inhaler to a subject suffering from a condition or disease, which can be treated with treprostinil, such as asthma, pulmonary hypertension, peripheral venous insufficiency, or pulmonary fibrosis.

A daily dose per patient for treatment or pulmonary hypertension, or conditions associated with pulmonary hypertension may be in the range 25 μg to 250 mg; 0.5 μg to 2.5 mg, or 7 μg to 285 μg, per day per kilogram bodyweight. For example, an intravenous dose in the range 0.5 μg to 1.5 mg per kilogram bodyweight per day may conveniently be administered as an infusion of from 0.5 ng to 1.0 μg per kilogram bodyweight per minute. One possible dosage is 2.5 ng/kg/min, increased over 12 weeks by an amount of 2.50 ng/kg/min each week, until a target dose, such as 15 ng/kg/min, is reached. Infusion fluids suitable for this purpose contain, for example, from 10 ng to 1 μg per milliliter. Ampoules for injection contain, for example, from 0.1 μg to 1.0 mg and orally administrable unit dose formulations, such as tablets or capsules, contain, for example, from 0.1 to 100 mg, typically from 1 to 50 mg. For diagnostic purposes, a single unit dose formulation may be administered. In the case of physiologically acceptable salts, the weights indicated above refer to the weight of the active compound ion, that is, the ion derived from Treprostinil.

In the manufacture of a medicament or diagnostic aid according to the invention, hereinafter referred to as a “formulation,” treprostinil and/or its derivatives, and/or pharmaceutically acceptable salts thereof, may be admixed with, inter alia, an acceptable carrier. The carrier must, of course, be acceptable in the sense of being compatible with any other ingredients in the formulation and must not be deleterious to the subject. The carrier may be a solid or a liquid, or both, and is preferably formulated with the compound as a unit-dose formulation, for example, a tablet, which may contain from 0.05% to 95% by weight of the active compound. One or more of treprostinil or its derivatives, or pharmaceutically acceptable salts thereof, may be incorporated in the formulations of the invention, which may be prepared by any of the well-known pharmaceutical techniques for admixing the components.

The formulations of the invention include those suitable for parenteral (e.g., subcutaneous, intramuscular, intradermal, or intravenous), oral, inhalation (in solid and liquid forms), rectal, topical, buccal (e.g., sub-lingual) and transdermal administration, although the most suitable route in any given case may depend on the nature and severity of the condition being treated and on the nature of the particular form of Treprostinil, its derivative, or a pharmaceutically acceptable salt thereof.

Formulations of the present invention suitable for parenteral administration conveniently comprise sterile aqueous preparations of Treprostinil or its derivative, or a pharmaceutically acceptable salt thereof, where the preparations may be isotonic with the blood of the intended recipient. These preparations may be administered by means of subcutaneous injection, although administration may also be effected intravenously or by means of intramuscular or intradermal injection. Such preparations may conveniently be prepared by admixing the compound with water or a glycine or citrate buffer and rendering the resulting solution sterile and isotonic with the blood. Injectable formulations according to the invention may contain from 0.1 to 5% w/v of active compound and may be administered at a rate of 0.1 ml/min/kg. Alternatively, the invention may be administered at a rate of 0.625 to 50 ng/kg/min. Alternatively, the invention may be administered at a rate of 10 to 15 ng/kg/min.

Formulations suitable for oral administration may be presented in discrete units, such as capsules, cachets, lozenges, or tablets, each containing a predetermined amount of Treprostinil or its derivative, or a pharmaceutically acceptable salt thereof; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients).

Treprostinil can be administered by inhalation, which in the present context refers to the delivery of the active ingredient, or a combination of active ingredients, through a respiratory conduit, in which the subject in need of the active ingredient (s) through the subject's airway, such as the nose or mouth of the subject.

A metered dose inhaler, in the present context, means a device capable of delivering a measured or bolus dose of a respiratory drug, such as treprostinil, to the lungs. An example of the device Inhalation can be a pressurized metered dose inhaler, a device that produces aerosol clouds for inhalation from solutions and/or suspensions of respiratory drugs in chlorofluorocarbon (CFC) and/or hydrofluoroalkane (HFA) solutions. The inhalation device can also be a dry powder inhaler. In such a case, the respiratory drug is inhaled in a solid formulation, usually in the form of a powder with a particle size of less than 10 micrometers in diameter, or less than 5 micrometers in diameter.

The metered dose inhaler can be a fine mist inhaler (SMI), in which the aerosol cloud containing a respiratory drug can be generated by passing a solution containing the respiratory drug through a nozzle or a series of nozzles. Aerosol generation can be achieved in the SMI, for example, by a mechanical, electromechanical or thermomechanical process. Examples of fine mist inhalers include the Respimat® inhaler (Boeringer Ingelheim GmbH), the AERx® inhaler (Aradigm Corp.), the Mystic™ inhaler (Ventaira Pharmaceuticals, Inc), and the Aira™ inhaler (Chrysalis Technologies Incorporated). For a review of fine mist inhaler technology, see, for example, M. Hindle, The Drug Delivery Companies Report, Autumn/Winter 2004, p. 31-34. The aerosol for the SMI can be generated from a solution of the respiratory drug which also contains pharmaceutically acceptable excipients. In the present case, the respiratory drug is treprostinil, its derivative or a pharmaceutically acceptable salt thereof, which can be formulated in the SMI as a solution. The solution can be, for example, a solution of treprostinil in water, in ethanol, or a mixture thereof. Preferably, the diameter of the aerosol particles containing treprostinil is less than about 10 micrometers, or less than about 5 micrometers, or less than about 4 micrometers.

The concentration of treprostinil in an aerosolizable formulation, such as a solution, used in a metered dose inhaler can range from about 500 pg/ml to about 2500 pg/ml, or from about 800 pg/ml to about 2200 pg/ml, or from about 1000 pg/ml to about 2000 pg/ml. In the present invention, the dose of treprostinil administered using a metered dose inhaler in a single drive is from about 30 pg to about 90 pg, or from about 30 pg to about 60 pg.

The administration of treprostinil in a single actuation can be carried out in a limited number of breaths by a patient. For example, treprostinil can be given in 20 breaths or less, or in 10 breaths or less, or in 5 breaths or less. Preferably, treprostinil is administered in 3, 2 or 1 breaths. The total time of a single administration drive may be less than 5 minutes, or less than 1 minute, or less than 30 seconds. Treprostinil can be administered once per day, or several times per day.

In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising Treprostinil or its derivative, or a pharmaceutically acceptable salt thereof, in a flavored base, usually sucrose and acacia or tragacanth; and pastilles comprising the compound in an inert base such as gelatin and glycerin or sucrose and acacia.

Formulations suitable for rectal administration are preferably presented as unit dose suppositories. These may be prepared by admixing Treprostinil or its derivative, or a pharmaceutically acceptable salt thereof, with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include vaseline, lanoline, polyethylene glycols, alcohols, and combinations of two or more thereof. The active compound is generally present at a concentration of from 0.1 to 15% w/w, for example, from 0.5 to 2% w/w. Formulations for transdermal administration may be delivered by iontophoresis (see, for example, Pharmaceutical Research, 3(6): 318 (1986)) and typically take the form of an optionally buffered aqueous solution of Treprostinil or its derivative or salt or thereof. Suitable formulations comprise citrate or bis/tris buffer (pH 6) or ethanol/water and contain from 0.1 to 0.2M active ingredient.

In some embodiments, the method of treating pulmonary hypertension may further comprise administering at least one supplemental agent selected from the group consisting of sildenafil, tadalafil, calcium channel blockers (diltiazem, amlodipine, nifedipine), bosentan, sitaxsentan, ambrisentan, and pharmaceutically acceptable salts thereof. In some embodiments, supplemental agents can be included in the treprostinil formulation, and thus can be administered simultaneously with treprostinil using a metered dose inhaler. In some embodiments, supplemental agents can be administered separately from treprostinil. In some embodiments, the application of intravenous prostacyclin (flolan), intravenous iloprost, or intravenous or subcutaneous treprostinil can be administered, in addition to treprostinil administered via inhalation using a metered dose inhaler.

The compounds of the present invention are conveniently prepared by methods the same as or analogous to those described in U.S. Pat. Nos. 4,306,075, 6,528,688 and 6,441,245.

In certain kit embodiments, the Treprostinil or its derivative, or a pharmaceutically acceptable salt thereof, is in a form suitable for subcutaneous administration, continuous subcutaneous infusion, intravenously administration or inhalation. In other kit embodiments, the Treprostinil or its derivative, or a pharmaceutically acceptable salt thereof, is in an orally available form selected from the group consisting of tablets and capsules. In another kit embodiment, the effective amount of Treprostinil or its derivative, or a pharmaceutically acceptable salt thereof, is at least 1.0 ng/kg of body weight/min.

Formulations of the current invention may also be employed to normalize biomarkers associated with pulmonary disease. Pulmonary disease and affected cells or tissue are associated with varied concentrations of proteins and cellular compounds. These compounds provide biomarkers to assess the severity and course of disease. For example, matrix metalloproteinase 9 (MMP-9), angiopoetin-2 (Ang-2), vascular endothelium-derived growth factor (VEG-F), and platelet derived growth factor (PDGF) are associated with lung disease and can be used in this invention to monitor the course of treatment with treprostinil or other pharmaceutical agent.

In some aspects, mean pulmonary arterial pressure is monitored by a CardioMEMs unit or a device capable of monitoring mean pulmonary arterial pressure remotely, continuously and in real time. In one aspect, one exemplary effective system and sensor suitable for measurement of hemodynamic parameters is the CARDIOMEMS pressure sensor. As described by U.S. Pat. No. 7,699,059 entitled “Implantable Wireless Sensor” and U.S. Pat. No. 7,679,355 entitled “Communicating with an Implanted Wireless Sensor,” which patent publications, in their entireties, are hereby incorporated by reference into this application, these pressure sensors are MEMS-based pressure sensors that are configured to be implanted: in the pulmonary artery, more particularly in the distal pulmonary artery branch, with a RHC or as part of a graft, such as a AAA stent-graft, and the like. The CARDIOMEMS pressure sensor are further configured to be selectively energized with RF energy to return high-frequency, high-fidelity dynamic pressure information from a precisely-selected location within a patient's body. In one aspect, advantages of the CARDIOMEMS pressure sensor when used in therapeutic development are that: the system is wireless, the pressure sensor is non-invasive after initial implantation, the pressure sensor is small enough to be implanted in a desired range of lumens and locations within a patient, and the pressure sensor is permanent or can be implanted for prolonged durations.

Another advantage of the CARDIOMEMS pressure sensor is that it can make measurements during ambulatory activities away from the hospital that are more representative of living conditions of a patient who is going to use a therapeutic. Because the CARDIOMEMS pressure sensor is non-invasive after implantation, ambulatory use is provided and the CARDIOMEMS sensor can be selectively energized via an easy-to-use RF transmitter within an external, non-invasive device that energizes the sensor. In another aspect, the CARDIOMEMS pressure sensor is configured to communicate pressure data wirelessly to a node local to the patient that is configured to transmit the information over the network to the front end computer system with little or no involvement of the patient.

In some aspects aspect, the mean pulmonary arterial pressure can be remotely obtained from patients outside of the hospital setting. For example, the desired physiology information could be obtained via a wireless sensor that's implanted in the patient's body, such as the exemplary CARDIOMEMS pressure sensor implanted in the patient's pulmonary artery.

In some aspects, a starting dose of a pulmonary vasodilator (e.g.: treprostinil) is administered to a subject, wherein the effect of the pulmonary vasodilator on mean pulmonary arterial pressure is monitored. The mean pulmonary arterial pressure can be continuously monitored, remotely monitored or monitored at various intervals. Upon detecting a change or improvement (i.e.: decrease) in mean pulmonary arterial pressure, the dose of the pulmonary vasodilator may be increased or decreased depending upon the subject's response to the vasodilator until the mean pulmonary arterial pressure of the subject reaches a target level. In this way, the method of the claimed invention provides for patient specific dose optimization.

The disclosure of all publications cited above are expressly incorporated herein by reference in their entireties to the same extent as if each were incorporated by reference individually.

The examples described herein are illustrative of the present invention and are not intended to be limitations thereon. Different embodiments of the present invention have been described according to the present invention. Many modifications and variations may be made to the techniques described and illustrated herein without departing from the spirit and scope of the invention. Accordingly, it should be understood that the examples are illustrative only and are not limiting upon the scope of the invention.

### Pharmaceutical Compositions

Treprostinil may be provided in a form of a pharmaceutical composition, which may also comprise a pharmaceutically acceptable carrier, excipient, binder, diluent or the like. Such pharmaceutical composition may be manufactured by methods known in the art such as granulating, mixing, dissolving, encapsulating, lyophilizing, emulsifying or levigating processes, among others. The composition may be in the form of, for example, granules, powders, tablets, capsules, syrup, suppositories, injections, emulsions, elixirs, suspensions and solutions. The composition may be formulated for a number of different administration routes, such as, for oral administration, transmucosal administration, rectal administration, transdermal or subcutaneous administration, as well as intrathecal, intravenous, intramuscular, intraperitoneal, intranasal, intraocular or intraventricular injection. The treprostinil may be administered by any of the above routes, for example in a local rather than a systemic administration, including as an injection or as a sustained release formulation.

In one embodiment, the pharmaceutical composition can compromise a prodrug of treprostinil and a carrier, such as sterile water. In some embodiments, the prodrug of treprostinil is formulated for subcutaneous administration, and such formulation may or may not include m-cresol or another preservative.

The treprostinil described herein can be used to treat pulmonary hypertension. In some embodiments, the treprostinil can be used to treat PAH. In some embodiments, the treprostinil can be used to treat one or more of WHO Groups 1-5 pulmonary hypertension, e.g., Group 1 pulmonary arterial hypertension. In some embodiments, treprostinil can be used to treat pulmonary hypertension classified in multiple WHO Groups but sharing a common characteristic, e.g., responsive to vasodilators. Likewise, the treprostinil described herein can be used to treat any disease or condition for which treprostinil is indicated or useful. The treprostinil can be administered as the sole therapeutic agent or in addition to other active agents, including treprostinil. In certain embodiments, treprostinil can be administered in combination with one or more additional active agents. In some embodiments, such one or more additional active agents can also be administered together with treprostinil using a metered dose inhaler. Even in some embodiments, such one or more additional active agents can be administered separately from treprostinil. Particular additional active agents that can be administered in combination with treprostinil may depend on a particular disease or condition, for which treatment or prevention is administered treprostinil. In some cases, the additional active agent may be a cardiovascular agent such as a calcium channel blocker, a phosphodiesterase inhibitor, an endothelial antagonist, or an antiplatelet agent.

For oral, buccal, and sublingual administration, powders, suspensions, granules, tablets, pills, capsules, gelcaps, and caplets may be acceptable as solid dosage forms. These can be prepared, for example, by mixing one or more treprostinil prodrugs, or pharmaceutically acceptable salts thereof, with at least one additive or excipient such as a starch or other additive. Suitable additives or excipients may be sucrose, lactose, cellulose sugar, mannitol, maltitol, dextran, sorbitol, starch, agar, alginates, chitins, chitosans, pectins, tragacanth gum, gum arabic, gelatins, collagens, casein, albumin, synthetic or semi-synthetic polymers or glycerides, methyl cellulose, hydroxypropylmethyl-cellulose, and/or polyvinylpyrrolidone. Optionally, oral dosage forms may contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Additionally, dyestuffs or pigments may be added for identification. Tablets may be further treated with suitable coating materials known in the art.

Liquid dosage forms for oral administration may be in the form of pharmaceutically acceptable emulsions, syrups, elixirs, suspensions, slurries and solutions, which may contain an inactive diluent, such as water. Pharmaceutical formulations may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Preferably, the oil or fatty acid is non-volatile, including natural or synthetic oils, fatty acids, mono-, di- or tri-glycerides.

For injection, the pharmaceutical formulation may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these. The compounds may be formulated for parenteral administration by injection such as by bolus injection or continuous infusion. A unit dosage form for injection may be in ampoules or in multi-dose containers. Besides those representative dosage forms described above, pharmaceutically acceptable excipients and carriers are generally known to those skilled in the art and can be employed. Such excipients and carriers are described, for example, in “Remingtons Pharmaceutical Sciences” Mack Pub. Co., New Jersey (1991), which is incorporated herein by reference.

A treprostinil prodrug may be formulated in a formulation suitable for parenteral administration that may comprise sterile aqueous preparations of a treprostinil prodrug, or a pharmaceutically acceptable salt thereof, where the preparations may be isotonic with the blood of the intended recipient. These preparations may be administered by means of subcutaneous injection, although administration may also be effected intravenously or by means of intramuscular or intradermal injection. Such preparations may conveniently be prepared by admixing the compound with water or a glycine or citrate buffer and rendering the resulting solution sterile and isotonic with the blood. Injectable formulations may contain from 0.1 to 5% w/v based on weight of treprostinil in the prodrug and may be administered at a rate of 0.1 ml/min/kg. Alternatively, the prodrug may be administered at a rate of 0.625 to 50 ng/kg/min based on weight of treprostinil in the prodrug. Alternatively, the prodrug may be administered at a rate of 10 to 15 ng/kg/min based on weight of treprostinil in the prodrug.

In some embodiments, a concentration of a treprostinil prodrug in a formulation for parenteral administration, such as intravenous infusion or subcutaneous infusion (including continuous subcutaneous infusion), may be from 0.0005 to 30 mg/mL or from 0.0007 to 50 mg/mL or from 0.001 to 15 mg/mL or any value or subrange within these ranges. Exemplary concentrations may include 0.1 mg/mL, 1 mg/mL, 2.5 mg/mL, 5 mg/mL or 10 mg/mL.

In some embodiments, a formulation of a treprostinil prodrug for parenteral administration, such as intravenous infusion or subcutaneous infusion (including continuous subcutaneous infusion), may be prepared by admixing the prodrug with a vehicle, such as a buffer. In certain embodiments, the vehicle may be a phosphate containing vehicle, i.e., at least one phosphate salt, which may be for example, dibasic phosphate, such as sodium dibasic phosphate or potassium dibasic phosphate, or tribasic phosphate, such as sodium tribasic phosphate or potassium phosphate. In certain embodiments, the vehicle may also contain a halogen salt, such as a chloride salt, which may be, for example, sodium chloride or potassium chloride. The halogen salt, such as sodium chloride may be used to adjust tonicity of the vehicle. In certain embodiments, it may be preferred that a phosphate and a halogen salt have the same cation. For example, when a phosphate is sodium phosphate, such as sodium tribasic phosphate or sodium tribasic phosphate, a halogen salt may a sodium halogen salt such as sodium chloride. Similarly, when a phosphate is potassium phosphate, such as potassium tribasic phosphate or potassium tribasic phosphate, a halogen salt may a potassium halogen salt such as potassium chloride. A solvent in the vehicle may contain water. In certain embodiments, water may be the only solvent in the vehicle. Yet in certain embodiments, the vehicle may contain one or more additional solvent in addition to water. In some embodiments, an additional solvent may be a preservative, such as m-cresol.

Preferably, the vehicle is isotonic with blood of a patient, such as a human being. The term isotonic may mean that the osmolarity and ion concentrations of the vehicle match those of the patient, such as human being. Non-limiting example of vehicles include phosphate-buffered saline, which is a water-based salt solution containing disodium hydrogen phosphate, sodium chloride and, in some formulations, potassium chloride and potassium dihydrogen phosphate. Other examples may include a vehicle containing 20 mM disbasic sodium phosphate with 125 mM sodium chloride and a vehicle containing 15 mM sodium phosphate tribasic, 125 mM sodium chloride and 0.3% w/w m-cresol.

### Methods of Treatment

One aspect of the present disclosure is directed to a method of treating pulmonary hypertension comprising, consisting or consisting essentially of monitoring mean pulmonary arterial pressure in a subject. The method further comprises, consists of, or consists essentially of administering to the subject a first therapeutically effective amount of a pulmonary vasodilator or an ester or salt thereof when mean pulmonary arterial pressure exceeds a threshold value in the subject. The method further comprises, consists of or consists essentially of administering an increasing dose of the pulmonary vasodilator or ester or salt thereof until mean pulmonary arterial pressure is reduced to a target level. In some aspects, the subject is a human.

In some aspects of the present disclosure, the pulmonary vasodilator comprises, consists of or consists essentially of a prostanoid or prostacyclin. In yet another aspect, the pulmonary vasodilator comprises, consists of or consists essentially of treprostinil.

In some aspects of the present disclosure, pulmonary arterial pressure in the subject is remotely monitored. In yet another aspect, the mean pulmonary arterial pressure is continuously monitored. In some aspects, mean pulmonary artery pressure is monitored using an implanted wireless pulmonary artery pressure sensor in the subject.

In some aspects of the present disclosure, the target level is less than 50 mm Hg, or less than 40 mm Hg, or less than 30 mm H, or less than 25 mm Hg. In yet further aspects, the threshold value is greater than 20 mm Hg, or greater than 30 mm Hg, or greater than 40 mm Hg. In some aspects, initial mean pulmonary arterial pressure is greater than 35 mmHg.

In some aspects of the present disclosure, the methods comprise, consist of or consist essentially of monitoring the right ventricle structure of the subject. In yet another aspect, the method further comprises, consists of or consists essentially of measuring right ventricular ejection fraction after a first period of time following administration of treprostinil, an ester or salt thereof.

In some aspects, ejection fraction is measured by cMRI. In yet another aspect, the method further comprises, consists of or consists essentially of measuring a ratio of stroke volume to end systolic volume. In yet another aspect, the method comprises, consists of or consists essentially of measuring a ratio of tricuspid annular plane systolic excursion to pulmonary artery systolic pressure.

In some aspects of the present disclosure, the subject has not been previously treated for pulmonary hypertension or has received an endothelin receptor antagonist and/or a phosphodiesterase type 5 inhibitor for less than 24 months, or less than 18 months, or less than 12 months, or less than 6 months, or less than 3 months, or less than 1 month.

In some aspects of the present disclosure, administering comprises, consists of or consists essentially of administering intravenously. In yet another aspect, administering comprises, consists of or consists essentially of administering by inhalation. In yet another aspect, administering comprises, consists of or consists essentially of administering an oral formulation.

Administration may be performed via a route described above, or, for example, orally, intravenously, intra-arterial, intramuscularly, intranasally, rectally, vaginally, or subcutaneously. In some embodiments, the composition is administered by an injection. In some embodiments, the administering is performed orally. In some embodiments, the administering is performed subcutaneously.

In some embodiments, said administering results in no or less pain at a site of the injection compared to administering treprostinil. Pain, or the reduction thereof, may be assessed by any medically recognized method known in the art, for example, numerical rating scale (NRS), visual analog scale (VAS, i.e., Wong-Baker Pain Scale), the FLACC scale, the CRIES Scale, the COMFORT Scale, the McGill Pain Scale, the Manoski Scale, or other categorical scales. In comparison to injection of treprostinil, the pain upon injection of the prodrug results in about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% less pain, as measured by a medically recognized method.

The subject treated may be a human, canine, feline, aves, non-human primate, bovine, or equine. In some embodiments, the subject is a human. In some embodiments, the subject is a human uncooperative or fearful of injections, for example, a pediatric or demented geriatric subject.

Embodiments described herein are further illustrated by, though in no way limited to, the following working examples.

### Example 1

### Methods

ARTISAN (Afterload Reduction To Improve Right Ventricular Structure And Function) clinical trial, NCT 05203510, is a prospective, multicenter, open-label study to evaluate the effect of early and rapid treprostinil therapy to reduce mPAP and reverse RV remodeling in PAH. Subjects will be PAH treatment naïve or received endothelin receptor antagonist and/or phosphodiesterase type 5 inhibitor therapy less than 6 months, mPAP>35 mmHg, WHO functional class II or III. After initiating parenteral treprostinil with rapid dose titration based on mPAP assessment, subjects may transition to oral treprostinil while continuing upward dose titration to further reduce mPAP. mPAP will be closely monitored using the CardioMEMS HF System, while RV structure and function will be monitored with cardiac magnetic resonance imaging (cMRI) and echocardiography.

### Results

Approximately 50 subjects will be enrolled. Primary endpoint is change in RV ejection fraction from baseline to Month 12, measured by cMRI. Key secondary endpoints include mPAP, clinical improvement, ratio of stroke volume to end systolic volume measured by cMRI, ratio of tricuspid annular plane systolic excursion to pulmonary artery systolic pressure measured by echocardiography, and survival through Month 36.

The major mechanism of action of treprostinil is direct vasodilation of pulmonary artery vascular beds and inhibition of smooth muscle cell proliferation. The mechanism of anti-remodeling action of treprostinil in human pulmonary arterial smooth muscle cells may benefit patients with PAH and may reduce pulmonary arterial wall remodeling (PloS One. 2018;13(11):c0205195). Following primary treatment with early Treprostinil with rapid dose titration to reduce mean pulmonary artery pressure (mPAP) below 35 mmHg with the aim of mPAP normalization and monitoring using the CardioMEMS HF system, subjects may see significant improvements in RV function as evaluated by primary endpoints. Normalizing mPAP and a reversal or improvement of RV function as measured by RV ejection fraction may result in increased survival and a more positive prognosis compared to untreated controls. These results will indicate that mPAP lowering through the use of pulmonary vasodilators may be a novel therapeutic approach to improving patient survival and prognosis. Specifically, the effect of pulmonary vasodilators monitored in real time via the use of CardioMEMS HF systems may be an effective strategy in mPAP lowering resulting in reverse RV remodeling and an improvement in survival in populations suffering from pulmonary hypertension. (Life, 2023; 13, 1202; see also Heart 2023; 0:1-7).

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

What is claimed is:

1. A method of treating pulmonary hypertension comprising
monitoring mean pulmonary arterial pressure in a subject;
administering to the subject a first therapeutically effective amount of a pulmonary vasodilator or an ester or salt thereof when mean pulmonary arterial pressure exceeds a threshold value in the subject;
administering an increasing dose of the pulmonary vasodilator or ester or salt thereof until mean pulmonary arterial pressure is reduced to a target level.

2. The method of claim 1, wherein the pulmonary vasodilator is a prostanoid or prostacyclin.

3. The method of claim 1, wherein the pulmonary vasodilator is treprostinil.

4. The method of claim 1, wherein mean pulmonary arterial pressure in the subject is remotely monitored.

5. The method of claim 1, wherein the mean pulmonary arterial pressure is continuously monitored.

6. The method of claim 1, wherein mean pulmonary artery pressure is monitored using an implanted wireless pulmonary artery pressure sensor in the subject.

7. The method of claim 1, wherein the target level is less than 50 mm Hg, or less than 40 mm Hg, or less than 30 mm H, or less than 25 mm Hg.

8. The method of claim 1, wherein the threshold value is greater than 20 mm Hg, or greater than 30 mm Hg, or greater than 40 mm Hg.

9. The method of claim 1, further comprising monitoring right ventricle structure of the subject.

10. The method of claim 1, further comprising measuring right ventricular ejection fraction after a first period of time following administration of treprostinil, an ester or salt thereof.

11. The method of claim 10, wherein ejection fraction is measured by cMRI.

12. The method of claim 1, further comprising measuring a ratio of stroke volume to end systolic volume.

13. The method of claim 1, further comprising measuring a ratio of tricuspid annular plane systolic excursion to pulmonary artery systolic pressure.

14. The method of claim 1, wherein the subject has not been previously treated for pulmonary hypertension or has received an endothelin receptor antagonist and/or a phosphodiesterase type 5 inhibitor for less than 24 months, or less than 18 months, or less than 12 months, or less than 6 months, or less than 3 months, or less than 1 month.

15. The method of claim 1 wherein the initial mean pulmonary arterial pressure is greater than 35 mmHg.

16. The method of claim 1, wherein administering comprises administering intravenously.

17. The method of claim 1, wherein administering comprises administering by inhalation.

18. The method of claim 1, wherein administering comprises administering an oral formulation.

19. The method of claim 1, wherein the subject is a human.

20. The method of claim 1, wherein right ventricular ejection fraction does not change more than 15 mm Hg during administering.
